SEQUENCE LISTING (8 sequences)

SEQ ID NO: 1 - DNA, 42 bp, Artificial Sequence
  Description of Artificial Sequence Synthetic primer
gtccttagtgatattgaccagagtttcaacaaagtagctgaa

SEQ ID NO: 2 - DNA, 46 bp, Artificial Sequence
  Description of Artificial Sequence Synthetic primer
gtgccttcttccactcctttcagtttctcttgtagtctcattaaga

SEQ ID NO: 3 - DNA, 38 bp, Artificial Sequence
  Description of Artificial Sequence Synthetic oligonucleotide
caaagtagctgaatgtgtcttaatgagactacaagaga

SEQ ID NO: 4 - DNA, 92 bp, Artificial Sequence
  Description of Artificial Sequence Synthetic oligonucleotide
gtccttagtgatattgaccagagtttcaacaaagtagctgaatgtgtcttaatgagactacaagagaaactgaaaggagtggaagaaggcac

SEQ ID NO: 5 - DNA, 25 bp, Artificial Sequence
  Description of Artificial Sequence Synthetic primer
tccttagtgatattgaccagagttt

SEQ ID NO: 6 - DNA, 20 bp, Artificial Sequence
  Description of Artificial Sequence Synthetic primer
tgccttcttccactcctttc

SEQ ID NO: 7 - DNA, 13 bp, Artificial Sequence
  Description of Artificial Sequence Synthetic probe
gctgaatgtgtct

SEQ ID NO: 8 - DNA, 13 bp, Artificial Sequence
  Description of Artificial Sequence Synthetic probe
gctgaacgtgtct

(19) United States
(12) Patent Application Publication
(10) Pub. No.: US 2021/0239685 A1
(43) Pub. Date: Aug. 5, 2021

(54) METHODS OF ANALYZING DNA IN URINE

(71) Applicant: THE TRUSTEES OF INDIANA UNIVERSITY, Bloomington, IN (US)

(72) Inventor: Tim LAUTENSCHLAEGER, Carmel, IN (US)

(21) Appl. No.: 16/972,158

(22) PCT Filed: Jun. 27, 2019
(86) PCT No.: PCT/US19/39449

§ 371 Date: Dec. 4, 2020

Related U.S. Application Data
(60) Provisional application No. 62/690,492, filed on Jun. 27, 2018.

(51) Int. Cl. G01N 33/53; C12Q 1/6869; C12Q 1/686; C12N 15/10

(52) U.S. Cl. CPC G01N 33/5308; C12Q 1/6869; G01N 2800/54; C12N 15/1003; C12Q 1/686

(57) ABSTRACT

The present disclosure relates to methods of extracting DNA from urine and methods of analyzing DNA in a urine sample. Methods are provided for extracting ctDNA from a urine sample and analyzing the extracted ctDNA for mutations indicative of a disease. The disclosure also relates to compositions for use in such methods.

### CROSS REFERENCE TO RELATED APPLICATIONS

This application claims the benefit of U.S. Provisional Patent Application No. 62/690,492 filed on Jun. 27, 2018, the disclosure of which is hereby expressly incorporated by reference in its entirety.

### TECHNICAL FIELD

The present disclosure relates to methods of extracting DNA from urine and methods of analyzing DNA in a urine sample. The disclosure also relates to compositions for use in such methods.

### BACKGROUND AND SUMMARY OF THE DISCLOSURE

Many cancer patients, particularly those with limited cancer burden and early stage cancers, have undetectable circulating tumor DNA (ctDNA) using standard blood ctDNA tests. This severely limits the usefulness of ctDNA testing for molecular characterization of tumors, determining eligibility of patients for targeted therapies, or for early cancer detection and recurrence monitoring. Thus, there is critical need to develop a method with increased ctDNA sensitivity.

Typical blood-based ctDNA tests have a low sensitivity in patients with limited cancer burden at least partially due to the low amount of blood being sampled (typically 10-20 mL at one time point). Importantly, plasma cell free DNA (cfDNA) is partially cleared through urinary excretion. Urine collected over 24 hours represents filtration >10 times of a person's entire blood volume and potentially functions as a pooling repository for a day's worth of plasma ctDNA. Moreover, analysis of 24-hour urine potentially avoids sampling errors and susceptibility to temporal ctDNA fluctuations associated with a one-time blood collection. Notably, the fragment length distribution of transrenal DNA in urine suggests that there are 10 to 100 times more transrenal DNA molecules of 30-60 base pair (bp) length than there are of 100-200 bp length; the latter are typically assayed for mutation quantification. Mutations in small 30-60 bp DNA fragments are not easily quantifiable using previous ctDNA assays.

Circulating tumor DNA (ctDNA) assays can be used to determine the presence of mutations, enabling identification of eligible patients for targeted therapies. Moreover, recent non-small cell lung cancer studies indicate that ctDNA has potential as an early recurrence detection marker. However, many patients with early stage cancers are ctDNA negative using standard tests. This limited sensitivity of current ctDNA assays represents a critical barrier to progress. Therefore, the methods described herein demonstrate a novel urine-based ctDNA assay with increased sensitivity.

Described herein are improved cancer screening and monitoring methods using a patient urine sample. This disclosure demonstrates novel methods to address issues related to blood-based ctDNA tests, allowing mutation quantification in small DNA fragments from large volumes of urine. An improvement in ctDNA signal is achieved by both analyzing 24-hour urine samples and targeting mutations in very small (e.g., about 30-60 bp) DNA fragments as opposed to the current standard of a one-time small volume blood ctDNA test targeting longer DNA fragments (e.g., ≥100 bp). This approach can overcome the sensitivity barrier currently preventing the widespread use of ctDNA as cancer screening and monitoring tools.

Previously unexplored signal gain benefits of urine-based ctDNA analysis over blood-based analysis are described herein. ctDNA analysis of a 24-hour urine sample exploits the fact that urine is a filtrate of blood and in theory can contain an entire day's worth of certain blood contents. The methods herein described provide evidence of dramatic ctDNA detection improvements over current standard methods. Therefore, the sensitivity increase of the described ctDNA assay over current standard ctDNA assays has the potential to radically transform ctDNA research. The signal increase of the novel urine ctDNA assay over current ctDNA assays is at least one hundred-fold. Thus, this assay can reduce the need for invasive tissue biopsies, identify more patients eligible for targeted therapies, and ultimately lead to improved cancer screening and monitoring applications with the potential to reduce cancer morbidity and mortality. Thus, this high sensitivity assay has the potential to transform cancer healthcare through further reduction of invasive biopsies, identification of more patients eligible for targeted therapies, improved early cancer detection, and recurrence monitoring.

In one embodiment, a method of collecting and extracting ctDNA from a urine sample is described. The method comprises urine crossflow diafiltration, neutralization of PCR inhibitors, and removal of non-transrenal DNA. In another embodiment, a method of analyzing the extracted ctDNA for mutations is described. In another embodiment, a method is provided to detect a mutation in ctDNA using large volume urine analysis. In another embodiment, the method can more accurately detect mutated ctDNA than a standard blood ctDNA test.

In one embodiment, a method of detecting or monitoring circulating tumor DNA (ctDNA) in a patient urine sample is described, the method comprising: (i) processing a patient urine sample to concentrate the sample; (ii) extracting the ctDNA from the sample; and, (iii) analyzing the ctDNA in the sample. In one embodiment, the ctDNA comprises short DNA fragments of less than 100 base pair (bp)in length.

In one embodiment, the processing step comprises filtering the sample, dialyzing the sample, or combinations thereof. In one embodiment, the processing step comprises urine crossflow diafiltration. In one embodiment, the method further comprises neutralizing PCR inhibitors in the sample. In one embodiment, the method further comprises removing the non-transrenal DNA from the sample. In one embodiment, processing the patient urine sample and extracting the ctDNA occur in the same step. In one embodiment, removing the transrenal DNA from the sample can occur during the processing and/or extracting step. In one embodiment, the urine sample is a sample that has been collected from a patient for about 24 hours. In one embodiment, the DNA is extracted using a size selectivity method.

In one embodiment, the ctDNA is analyzed for mutations. In one embodiment, the mutation indicates a disease state in the patient. In one embodiment, the disease is cancer. In one embodiment, the disease is non-small cell lung carcinoma. In one embodiment, the ctDNA is analyzed by PCR. In one embodiment, the PCR is overlap extension PCR (OE PCR). In one embodiment, the PCR is digital droplet PCR (ddPCR). In one embodiment, the PCR is Emulsion PCR (EmPCR). In one embodiment, the ctDNA is analyzed for the presence of a tumor marker or a tumor recurrence marker. In one embodiment, the method comprises monitoring the patient for cancer progression. In one embodiment, the method comprises determining if the patient is eligible for a targeted cancer therapy. In one embodiment, the cancer is a non-metastatic cancer. In one embodiment, the extracted ctDNA comprises a short DNA fragment of about 30 to about 60 bp in length.

The various embodiments described in the numbered clauses below are applicable to any of the embodiments described in this “SUMMARY” section and the sections of the patent application titled “DETAILED DESCRIPTION OF ILLUSTRATIVE EMBODIMENTS” or “EXAMPLES” or in the “CLAIMS” appended to this application:

1. A method of detecting or monitoring circulating tumor DNA (ctDNA) in a patient urine sample comprising:
i. processing a patient urine sample to concentrate the sample;
ii. extracting the ctDNA from the sample; and
iii. analyzing the ctDNA in the sample wherein the ctDNA is a short DNA fragment of less than 100 base pair (bp).

2. The clause of claim 1, wherein the processing step comprises filtering the sample, dialyzing the sample, or combinations thereof

3. The clause of claim 1, wherein the processing step comprises urine crossflow diafiltration.

4. The clause of claim 1, further comprising neutralizing PCR inhibitors in the sample.

5. The clause of claim 1, further comprising removing the non-transrenal DNA from the sample.

6. The clause of claim 1, wherein processing the patient urine sample and extracting the ctDNA occur in the same step.

7. The clause of claim 1, wherein the urine sample is a sample that has been collected from a patient for about 24 hours.

8. The clause of claim 1, wherein the DNA is extracted using a size selectivity method.

9. The clause of claim 1, wherein the ctDNA is analyzed for mutations.

10. The clause of claim 9, wherein the mutation indicates a disease state in the patient.

11. The clause of claim 10, wherein the disease is cancer.

12. The clause of claim 10, wherein the disease is non-small cell lung carcinoma.

13. The clause of claim 9, wherein the ctDNA is analyzed by PCR.

14. The clause of claim 13 wherein the PCR is overlap extension PCR (OE PCR).

15. The clause of claim 13 wherein the PCR is digital droplet PCR (ddPCR) or Emulsion PCR (EmPCR).

16. The clause of claim 1, wherein the ctDNA is analyzed for the presence of a tumor marker or a tumor recurrence marker.

17. The clause of claim 1, comprising monitoring the patient for cancer progression.

18. The clause of claim 1, comprising determining if the patient is eligible for a targeted cancer therapy.

19. The clause of claim 11, wherein the cancer is a non-metastatic cancer.

20. The clause of claim 1, wherein the extracted ctDNA comprises short DNA fragments of about 30 to about 60 bp in length.

### BRIEF DESCRIPTION OF THE DRAWINGS

FIG. 1. shows that input DNA amount limits detection of low MAF ctDNAs.

FIG. 2. shows that urine filtration using PES membrane facilitates analysis of large urine volumes (here PES crossflow filtered 2500 mL of urine generate a 77-fold signal increase over unfiltered 30 mL of urine at 93% efficiency).

FIG. 3. shows that the overlap extension PCR elongates short DNA fragments so they become quantifiable using standard ddPCR. Overlap extension primers (forward orange and reverse green) bind to a region ≥15 bp on a short DNA fragment of interest (blue). After two PCR cycles, each of the red circled dsDNA PCR products (right side) includes one fully elongated single fragment. The fully elongated fragments can then be assayed in downstream assays (i.e. ddPCR). In summary, this reaction produces two fully elongated DNA fragments out of two originally short fragments.

FIG. 4. shows two cycle overlap extension PCR (OE) elongates short ATM wildtype or R3008C mutant DNA fragments (template), which then can be quantified using ddPCR (top block blue: mutant channel, bottom block green: wildtype channel). The elongation of mutant fragment is successful in a background of human plasma DNA.

FIG. 5. shows the addition of synthetic DNA to urine or plasma shows that extension PCR works reliably in complex backgrounds such as urine and plasma.

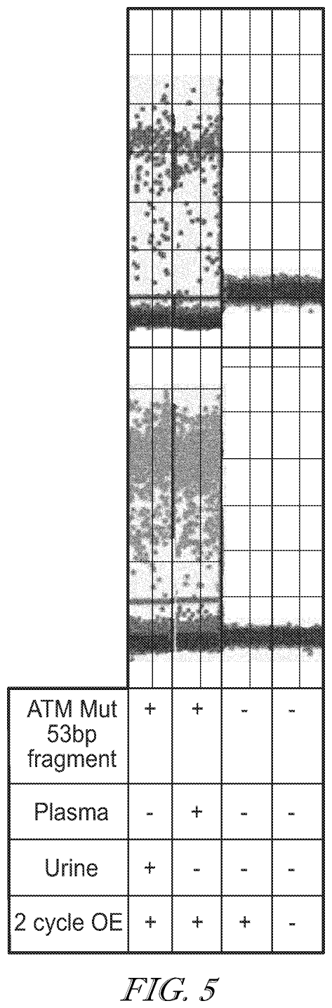

FIG. 6. shows the comparison of R3008C mutant ATM ctDNA signal obtained using our new assay vs. a standard assay analyzing matched blood and urine samples. Numbers in table are fold changes of signal relative to a 10 mL standard blood assay.

FIG. 7. shows the effectiveness of urine cfDNA preservation methods. If no preservative is used, >90% of cfDNA signal (33 bp ALB assay) is lost after three days of urine storage at room temperature. A commercial urine ctDNA preservative (Norgen) performed best keeping signal stable for 14 days.

FIG. 8. is a table showing urine processing using tangential flow polyethersulfone (PES) membrane diafiltration prior to DNA extraction increases ctDNA signal over direct DNA extraction of the same urine sample.

FIG. 9. shows OE temperature ramp speed increases separation between positive (box) and negative control signal (black band).

FIG. 10. shows ligation based DNA elongation.

FIG. 11. shows primer increase during ddPCR increases separation between mutant (blue) and control signal (black bands above x-axis).

### DETAILED DESCRIPTION OF THE INVENTION

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

The present disclosure is generally related to methods of collecting, extracting, and analyzing DNA in a urine sample. In particular, methods and compositions are provided in accordance with the present invention for collecting, extracting, and analyzing cell-free DNA (cfDNA) in a sample or circulating tumor DNA (ctDNA) in a sample. As described herein, the method can increase the sensitivity of detection of circulating tumor DNA (or DNA of another origin, i.e. infection or fetal) by a factor of at least one hundred or several hundred. For example, the methods described herein can be useful for high sensitivity infection screening (e.g., viral: HIV, Hepatitis, etc.) and high sensitivity noninvasive prenatal testing (e.g., gender testing).

As described herein, the method can detect several hundred-fold more cancer genome copies than a blood-based assay. Urine is a filtrate of plasma and 24-hour urine collections are routinely used clinically for a variety of indications. A 24-hour urine collection is typically the result of greater than 10 times the filtration of a person's entire blood volume. Thus, a 24-hour urine collection represents certain plasma contents over 24 hours, and potentially includes more ctDNA than a 10 mL blood sample. Previous studies have focused on patients with widely metastatic disease, who typically have several magnitudes more ctDNA than non-metastatic patients. Previous studies have not incorporated large volume urine analysis or evaluated the sensitivity gains associated with small DNA fragment analysis vs. standard size DNA fragments analysis. The methods described herein can increase the sensitivity of ctDNA detection: (i) collection and processing of large volumes of urine (24-hour collection) and (ii) recovery and analysis of highly abundant small transrenal DNA fragments (e.g., 30-60 bp). This is compared to a one-time 10 mL blood collection with analysis of larger DNA fragments (e.g., 100-200 bp) typically targeted by standard blood-based ctDNA analysis.

In one embodiment, a method of detecting or monitoring circulating tumor DNA (ctDNA) in a patient urine sample is described comprising: (i) processing a patient urine sample to concentrate the sample; (ii) extracting the ctDNA from the sample; and (iii) analyzing the ctDNA in the sample. In one embodiment, the ctDNA is a short DNA fragment of less than 100 base pair (bp). In one embodiment, the ctDNA can be a short DNA fragment of about 30 to about 60 bp.

In one embodiment, the processing step comprises filtering the sample, dialyzing the sample, or combinations thereof. For example, the processing step can be urine crossflow diafiltration. In one embodiment, a combination of filtration and dialysis (diafiltration) can be used to concentrate very large amounts of urine down to mL sized samples, which removes molecules that interfere with or prevent common DNA analysis methods. For example, the urine sample may be concentrated to a sample size of 1 mL, 0.25 mL, 0.5 mL, 0.75 mL, 1.5 mL, 2 mL, 2.5 mL, 3 mL, 3.5 mL, 4 mL, 4.5 mL, 5 mL, 6 mL, 7 mL, 8 mL, 9 mL, 10 mL, 12 mL, 15 mL, 20 mL, 25 mL, 30 mL, 45 mL, 50 mL, 75 mL, 100 mL, 150 mL, 200 mL, 250 mL, 500 mL, or similar. As described herein, any method of concentrating a sample may be employed. As used herein, diafiltration is a process the removes or separates components (e.g., permeable molecules like salts, proteins, solvents, etc.) of a solution.

In one embodiment, the method further comprises neutralizing PCR inhibitors in the sample. In one embodiment, the method further comprises removing the non-transrenal DNA from the sample. In some embodiments, processing the patient urine sample and extracting the ctDNA occur in the same step. In another embodiment, the method provides, wherein the urine is diluted with a salt-free solution prior to extraction. Further the salt-free solution can be deionized water. In one embodiment, the DNA can be extracted using a size selectivity method. In one embodiment, the urine sample is a sample that has been collected from a patient for about 24 hours. Methods are provided for the detection of ctDNA in an extended collection urine sample, e.g., a 24-hour urine sample. For example, the method can include a 24 hour urine collection in lieu of the standard one-time blood draw or urine collection, thereby obtaining more DNA for analysis.

In one embodiment, an innovative two-pronged approach is disclosed including, (1) recovery of DNA from large volume 24-hour urine collections and (2) mutation detection in very small DNA fragments representing the majority of ctDNA molecules in urine. In another embodiment, a method is disclosed comprising a workflow for DNA recovery from large amounts of urine using crossflow polyethersulfone (PES) membrane diafiltration, making DNA from urine suitable for common downstream applications including PCR and next generation sequencing (NGS). In another embodiment, a method is disclosed comprising using an overlap extension (OE) PCR based very short DNA fragment preparation method, which will elongate fragments, allowing routine mutation quantification using well establish platforms.

In one embodiment, the ctDNA is analyzed for mutations. For example, the mutation may indicate a disease state in the patient. In one embodiment, the disease is cancer, e.g., the cancer may be non-small cell lung carcinoma. In one embodiment, the cancer is a non-metastatic cancer. In one embodiment, the ctDNA is analyzed by PCR, e.g., overlap extension PCR (OE PCR), emulsion PCR (EmPCR), or digital droplet PCR (ddPCR) may be used. In one embodiment, the ctDNA is analyzed for the presence of a tumor marker or a tumor recurrence marker. In one embodiment, a DNA elongation method can be employed that transforms small DNA molecules into longer DNA fragments that can be analyzed using standard methods, thus allowing the analysis of more DNA molecules of interest than otherwise possible. In one embodiment, the method further comprises monitoring the patient for cancer progression. In one embodiment, the method further comprises determining if the patient is eligible for a targeted cancer therapy.

As described herein, circulating tumor DNA (ctDNA) is tumor-derived fragmented DNA that is not associated with cells. Cell-free DNA (cfDNA) refers to DNA that is freely circulating in the bloodstream, but is not necessarily of tumor origin.

As described herein, a genetic marker is a specific sequence of DNA at a known location on a chromosome. Examples of genetic markers may include single polymorphism nucleotides (SNPs) and microsatellites. A genetic marker of susceptibility is a specific change in a person's DNA that makes the person more likely to develop certain diseases such as cancer.

As described herein, a biomarker, or molecular marker, is a biological molecule found in blood, urine, other body fluids, or tissues that is a sign of a normal or abnormal process, or of a condition or disease. A biomarker may be used to see how well the body responds to a treatment for a disease or condition. Many specific biomarkers have been well characterized and repeatedly shown to correctly predict relevant clinical outcomes across a variety of treatments and populations.

As described herein, tumor markers are substances that are produced by cancer cells or by other cells of the body in response to cancer or certain benign (noncancerous) conditions. Most tumor markers are made by normal cells as well as by cancer cells; however, they are produced at much higher levels in cancerous conditions. These substances can be found in the blood, urine, stool, tumor tissue, or other tissues or bodily fluids of some patients with cancer. Tumor markers may include, e.g., proteins, DNA, RNA, etc. For example, certain patterns of gene expression and changes to DNA can be used as tumor markers. A tumor recurrence marker is a tumor marker used in monitoring the tumor recurrence in a patient.

As described herein, the mutant allele fractions (MAFs), also called ‘mutation dose’, represent the number of mutant reads divided by the total number of reads at a specific genomic position. In some scenarios, the MAFs of certain genes may have important clinical implications. The mutant-allele fraction heterogeneity may relate to overall survival in cancer patients.

The methods described herein may be useful to determine specific molecules, e.g., a tumor marker or a tumor recurrence marker, to predict the risk of tumor relapse after a specific treatment or curative surgery. In one embodiment, ctDNA from a patient sample is analyzed for the presence of a tumor marker, a tumor recurrence marker, a genetic marker, and/or, a biomarker. In one embodiment, the mutant allele fraction of a specific gene is determined. In one embodiment, the heterogeneity of the mutant allele fraction is determined.

In accordance with the invention, “patient” may refer to a human or an animal. Accordingly, the methods and compositions disclosed herein can be used for both human clinical medicine and veterinary applications. Thus, as described herein, a “patient” can be a human or, in the case of veterinary applications, the patient can be a laboratory, an agricultural, a domestic, or a wild animal. In various aspects, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale. Exemplary patients include cancer patients, post-operative patients, transplant patients, patients undergoing chemotherapy, immunosuppressed patients, and the like. In one embodiment, the sample is obtained from a patient. In another embodiment, the sample is a urine sample from a patient. The samples can be prepared for testing as described herein.

In various embodiments, the ctDNA may be derived from a carcinoma, a sarcoma, a lymphoma, a melanoma, a mesothelioma, a nasopharyngeal carcinoma, a leukemia, an adenocarcinoma, or a myeloma. In other embodiments, the DNA may be from a lung cancer, bone cancer, pancreatic cancer, hepatobiliary cancer, cancer of the gallbladder, skin cancer, cancer of the head, cancer of the neck, cutaneous melanoma, intraocular melanoma, uterine cancer, ovarian cancer, endometrial cancer, rectal cancer, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, prostate cancer, penile cancer, testicular cancer, pancreatic endocrine cancer, carcinoid cancer, retinoblastomas, Hodgkin's lymphoma, non-Hodgkin's lymphomachronic leukemia, acute leukemia, a lymphocytic lymphoma, mesothelioma, cancer of the bladder, Burkitt's lymphoma, cancer of the ureter, cancer of the kidney, renal cell carcinoma, carcinoma of the renal pelvis, a neoplasm of the central nervous system (CNS), primary CNS lymphoma, a spinal axis tumor, a brain stem glioma, a pituitary adenoma, or an adenocarcinoma. In one embodiment, the ctDNA is derived from a non-small-cell lung carcinoma.

In one embodiment, a method is provided for extracting DNA from a urine sample comprising collecting a urine sample from a patient and extracting the DNA from the urine sample. In one embodiment, the urine sample is filtered after collection. In one embodiment, the urine is dialyzed after collection. In another embodiment, the urine is concentrated after collection, e.g., by dialyzing and/or filtering the sample. In another embodiment, the method comprises detecting DNA in the urine sample. The sample may be collected over a period of time (e.g., 24 hour collection), instead of a one-time sample collection. For example, the sample may be collected for about 6 hours, about 12 hours, about 18 hours, about 24 hours, about 30 hours, about 36 hours, about 48 hours, about 60 hours, or about 72 hours. In further embodiments, the method includes the detection of small DNA fragments. As described herein, the method results in an increase in sensitivity for DNA detection by a factor of up to one hundred or several hundred. In one embodiment, the DNA analysis results in an increase in signal over standard mutations assays, e.g., the method may result in a 2, 5, 10, 20, 30, 40, 45, 50, 60, 70, 75, 80, 85, 90, 95, 100, 110, 120, 150, 200, 250, 300, 350, 400, 450, or 500-fold increase in signal over standard mutation assays. In one embodiment, the method may provide a greater than 500-fold increase in signal over standard assays.

DNA found in urine consists of both transrenal and nontransrenal fractions and the amounts of non transrenal DNA extracted from large sample volumes can overwhelm sensitive assays intended to quantify mutations in transrenal DNA. To enrich for ctDNA found only in transrenal DNA, the longer, non transrenal DNA fragments in the sample may be reduced or eliminated using size selection methods. Various size selection methods can be employed and are well known to those having ordinary skill in the art.

In one embodiment, the urine sample is collected in a urine collection container. In another embodiment, the DNA is extracted using a size selectivity method. In another embodiment, the size selectivity method is a membrane with a pore size that allows DNA to pass through ranging in size up to about 60 bp in length or up to about 100 bp in length. For example, the pore size may allow DNA to pass through in a size of up to about 5, 10, 15, 20, 25, 30, 35, 40, 45, 50, 55, 60, 65, 70, 75, 80, 85, 90, 95, or 100 bp in length. In one embodiment, the pore size may allow DNA to pass through in a size of up to about 110, 120, 130, 140, 150, 160, 170, 180, 190, 200, 210, 220, 230, 240, or 250 bp in length or similar. For example, the pore size may be 0.12 μm, 0.2 μm, or 0.45 μm. In one embodiment, the pore size is selected from 5 nm, 10 nm, 12 nm, 35 nm, 0.1 μm, 0.12 μm, 0.15 μm, 0.2 μm, 0.22 μm, 0.4 μm, and 0.45 μm. The pore size used will depend on the size of the DNA molecules desired in the sample. In various embodiments, the desired size of the DNA molecule for analysis are about 30-60, 10-60, 10-20, 10-30, 10-40, 10-50, 20-60, 20-50, 20-40, 20-30, 30-50, 30-40, 25-75, or 30-100 bp. In one embodiment, the DNA molecules for analysis may be less than 100 bp in length. For example, the DNA molecules for analysis may be less than 100 bp, less than 75 bp, less than 60 bp, or less than 50 bp in length.

In one embodiment, a method is developed to extract DNA from large volumes of urine suitable for downstream mutation analysis. As herein described, a three-pronged approach may be employed to large volume urine ctDNA sample processing and analysis, consisting of urine crossflow diafiltration, neutralization of PCR inhibitors, and removal of non-transrenal DNA. In one embodiment, a method of analyzing transrenal cell-free DNA (cfDNA) from 24-hour urine samples (≤3L) is described. In another embodiment, the sample collection can take place over a period of time greater than eight (8) hours and less than twenty-five (25) hours. In other embodiments, the sample of urine from a patient may range from greater than 1 L to 4 L of urine. In another embodiment, the urine sample may be between 0.5 L and 3.5 L of urine. In another embodiment, the urine sample is greater than 0.5 L of urine.

In one embodiment, crossflow diafiltration combines dialysis for removal of soluble PCR inhibitors with concentration of urine prior to ctDNA extraction. Useful polyethersulfone (PES) membrane pore sizes are described herein to maximize recovery of, e.g., 30-60 bp DNA fragments without significant loss in recovery of 100-200 bp fragments while ensuring optimal removal of PCR inhibitors. In another embodiment the membrane to separate the ctDNA from the rest of the fluid is comprised of a hydrophilic membrane. In another embodiment, the membrane is a hydrophilic membrane. In another embodiment, the extracted ctDNA desired length is between 25 and 75 bp. In another embodiment the extracted ctDNA is at least 25 bp in length. In another embodiment, the extracted ctDNA is greater than 100 bp in length. In another embodiment, the desired extracted ctDNA is between 26 and 64 bp in length. Moreover, alterations in crossflow diafiltration parameters, such as addition of significant amounts of a salt-less solution, can further improve DNA desalting. In one embodiment, the salt-less solution is deionized water. In one embodiment the amount of deionized water added to the urine sample is a ratio of 1:1 urine to deionized water. In another embodiment the urine sample is diluted by about 50% with deionized water. In another embodiment the urine sample is diluted with up to 6 L of deionized water. For example, the urine may be diluted ata ratio of 0.5:1, 1:1, 1:2, 1:3, 1:4, 1:5, or 1:10. Current commercial urine DNA extraction kits re-introduce PCR inhibitors via salts present in the buffers. To address this, PCR additives, e.g., bovine serum albumin and the like, may be used to neutralize PCR inhibition introduced by the DNA extraction process.

In one embodiment, the method is used to extract DNA and detect a disease. In a further embodiment, the DNA extracted is ctDNA and the disease is cancer. In some embodiments, the cancer comprises a primary tumor. In yet other embodiments, the cancer comprises non-metastatic tumor cells. In yet other embodiments, the cancer comprises metastatic tumor cells.

In one embodiment, detecting the ctDNA in the sample comprises quantifying the copy number of a gene in the ctDNA sample. In one embodiment, detecting the ctDNA in the sample comprises detecting a mutation in the ctDNA sample. In some embodiments, the gene copy number is quantified per ml of sample. The methods described herein can be used to detect or identify specific nucleic acid sequences in a DNA sample. Techniques for isolation of DNA are well-known in the art. Methods for isolating DNA are described in Sambrook et al., “Molecular Cloning: A Laboratory Manual”, 3rd Edition, Cold Spring Harbor Laboratory Press, (2001), incorporated herein by reference.

In one embodiment, a method of analyzing the ctDNA for a mutation is provided including: providing primer(s) and/or probe(s), amplifying the ctDNA, sequencing the ctDNA, and analyzing the sequenced ctDNA for mutations. In various embodiments, the mutation is indicative of a disease, e.g., cancer. In one embodiment, the ctDNA may be analyzed for the presence of a specific mutant allele fraction, a genetic marker, a biomarker, a tumor marker, or a tumor recurrence marker. In one embodiment, the amplification method is a PCR method, such as OE PCR or ddPCR.

In the methods herein described, DNA may be detected and/or quantified using any DNA detection method known in the art. In one embodiment, the nucleic acid may be detected using conventional polymerase chain reaction (PCR) methods. In one embodiment, the nucleic acid may be detected using conventional polymerase chain reaction (PCR), quantitative PCR (qPCR), overlap extension PCR (OE PCR), Emulsion PCR (EmPCR), or digital PCR (dPCR). As described herein, PCR techniques may be used to amplify specific, target DNA fragments from low quantities of source DNA or RNA (for example, after a reverse transcription step to produce complementary DNA (cDNA), or detection of small fragment ctDNAs in a sample). When performing conventional PCR, the final concentration of template is proportional to the starting copy number and the number of amplification cycles. In one embodiment, a given number of reactions is performed on a single sample and the result is an analysis of fragment sizes or, for quantitative real-time PCR (qPCR), the analysis is an estimate of the concentration of the target sequences in the reaction-based on the number of cycles required to reach a quantification cycle (Cq).

For qPCR methods, a fluorescent reporter dye is used as an indirect measure of the amount of nucleic acid present during each amplification cycle. The increase in fluorescent signal is directly proportional to the quantity of exponentially accumulating PCR product molecules (amplicons) produced during the repeating phases of the reaction. Reporter molecules may be categorized as; double-stranded DNA (dsDNA) binding dyes, dyes conjugated to primers, or additional dye-conjugated oligonucleotides, referred to as probes. The use of a dsDNA-binding dye, such as SYBR® Green I, represents the simplest form of detection chemistry. When free in solution or with only single-stranded DNA (ssDNA) present, SYBR Green I dye emits light at low signal intensity. As the PCR progresses and the quantity of dsDNA increases, more dye binds to the amplicons and hence, the signal intensity increases. Alternatively, a probe (or combination of two depending on the detection chemistry) can add a level of detection specificity beyond the dsDNA-binding dye, since it binds to a specific region of the template that is located between the primers. The most commonly used probe format is the Dual-Labeled Probe (DLP; also referred to as a Hydrolysis or TaqMan® Probe). The DLP is an oligonucleotide with a 5′ fluorescent label, e.g., 6-FAM™ and a 3′ quenching molecule, such as one of the dark quenchers e.g., BHQ®1 or OQ™ (see Quantitative PCR and Digital PCR Detection Methods). These probes are designed to hybridize to the template between the two primers and are used in conjunction with a DNA polymerase that has 5′ to 3′ exonuclease activity.

For digital PCR (dPCR), the sample can be diluted and separated into a large number of reaction chambers or partitions. In various embodiments, each partition contains either one copy of the target DNA or no copies of the target DNA. In some embodiments, the partition may contain one or more copies of the target DNA. In some embodiments, the partition may contain two or more copies of the target DNA. The number of reaction chambers or partitions varies between systems, from several thousand to millions. The PCR is then performed in each partition and the amplicon detected using a fluorescent label such that the collected data are a series of positive and negative results.

In one embodiment, the methods described herein may include droplet digital PCR (ddPCR) technology. ddPCR is a method for performing digital PCR that is based on water-oil emulsion droplet technology. For example, a sample is fractionated into thousands of droplets (e.g., 10,000, 15,000, 20,000, 25,000, 30,000, 40,000, or 50,000 droplets, or more depending on the reaction to be performed), and PCR amplification of the template molecules occurs in each individual droplet. The droplets for use in ddPCR are typically nanoliter-sized droplets. ddPCR has a small sample requirement reducing cost and preserving samples.

As described herein, for methods employing ddPCR, the sample(s) may be partitioned into 20,000 nanoliter-sized droplets. This partitioning allows the measurement of thousands of independent amplification events within a single sample. ddPCR technology uses reagents and workflows similar to those used for most standard TaqMan probe-based assays. ddPCR allows the detection of rare DNA target copies, allows the determination of copy number variation, and allows the measurement of gene expression levels with high accuracy and sensitivity. Digital PCR is an end-point PCR method that is used for absolute quantification and for analysis of minority sequences against a background of similar majority sequences, e.g., quantification of somatic mutations. When using this technique, the sample is taken to limiting dilution and the number of positive and negative reactions is used to determine a precise measurement of target concentration. The digital PCR (dPCR) methods may be employed using emulsion beads (e.g., Bio-Rad QX100™ Droplet Digital™ PCR, ddPCR™ system and RainDance Technologies' RainDrop™ instrument). In an alternative format, the reactions may be run on integrated fluidic circuits (chips). These chips have integrated chambers and valves for partitioning samples and reaction reagents (e.g., BioMark™, Fluidigm).

For overlap extension PCR (OE-PCR) the method may be used for eample, for DNA elongation, to insert specific mutations at specific points in a sequence, or to splice smaller DNA fragments into a larger polynucleotide. In one embodiment, a method is described for detection of mutations in very small DNA fragments. Small fragment DNA elongation can be accomplished using a variation of overlap extension (OE) PCR. Overlap extension (OE) PCR-based DNA fragment elongation methods are described herein. For example, two PCR cycles or more using extension primers may be employed to elongate a fragment of interest while also limiting PCR-errors. In one embodiment, very short DNA fragments are targeted (e.g., 30-60 bp, or as previously described herein) with both primers having 15 bp or more overlap with the DNA template. For example, the primers may have an overlap with the DNA molecule template that is 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, or more. Given that the exact DNA fragment sizes harboring a mutation of interest may be unknown in a clinical sample, extension primers are designed using the gene's native DNA sequence to extend a wide range of short DNA fragments. PCR parameters including primer concentration, concentration of PCR additives, PCR annealing temperatures, and temperature ramp speeds are analyzed for contribution to maximizing elongation efficiency. In another embodiment, a method of analyzing the ctDNA for a mutation is provided including: providing primer(s) and/or probe(s), amplifying the ctDNA, sequencing the ctDNA, and analyzing the sequenced ctDNA for mutations. In various embodiments, the mutation is indicative of a disease, e.g., cancer. In one embodiment, the amplification method is a PCR method, such as OE PCR, EmPCR, or ddPCR.

Emulsion PCR (EmPCR) may be used for template amplification, e.g., in multiple NGS-based sequencing platforms. The basic principle of emPCR is dilution and compartmentalization of template molecules in water droplets in a water-in-oil emulsion. Ideally, the dilution is to a degree where each droplet contains a single template molecule and functions as a micro-PCR reaction. As described herein, emulsion PCR can overcome possible OE PCR bias for elongation of ultra-low frequency mutations. Elongation efficiency and false positive mutation rates may be analyzed to determine optimal PCR conditions, utilizing in vitro systems of varying mutant and wildtype DNA fragment sizes and ratios, and modeling human urine, which contains DNA of differing fragment lengths.

Techniques for isolation of DNA are well-known in the art. In one embodiment, cells may be ruptured by using a detergent or a solvent, such as phenolchloroform. In another embodiment, cells remain intact and cell-free DNA may be extracted. DNA may be separated from other components in the sample by physical methods including, but not limited to, centrifugation, pressure techniques, or by using a substance with affinity for DNA, such as, for example, silica beads. After sufficient washing, the isolated DNA may be suspended in either water or a buffer. In other embodiments, commercial kits are available, such as Quiagen™, Nuclisensm™, and Wizard™ (Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

In various embodiments described herein, primers and/or probes are used for amplification of the target DNA are oligonucleotides from about ten to about one hundred, more typically from about ten to about thirty or about twenty to about twenty-five base pairs long, but any suitable sequence length can be used. In illustrative embodiments, the primers and probes may be double-stranded or single-stranded, but the primers and probes are typically single-stranded. The primers and probes described herein are capable of specific hybridization, under appropriate hybridization conditions (e.g., appropriate buffer, ionic strength, temperature, formamide, or MgCl₂ concentrations), to a region of the target DNA. The primers and probes described herein may be designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference.

Also within the scope of the invention are nucleic acids complementary to the probes and primers described herein, and those that hybridize to the nucleic acids described herein or those that hybridize to their complements under highly stringent conditions. In accordance with the invention “highly stringent conditions” means hybridization at 65° C. in 5×SSPE and 50% formamide, and washing at 65° C. in 0.5×SSPE. Conditions for low stringency and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

In some embodiments, also included are nucleic acid molecules having about 60%, about 70%, about 75%, about 80%, about 85%, about 90%, about 95%, at least 96%, at least 97%, at least 98%, or at least 99% homology to the probes and primers described herein. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software. In some embodiments, the percent identity can be determined along the full-length of the nucleic acid. As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

Techniques for synthesizing the probes and primers described herein are well-known in the art and include chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, Calif. or Integrated DNA Technologies, Skokie, Il.). Techniques for purifying or isolating the probes and primers described herein are well-known in the art. Such techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as restriction enzyme analysis or sequencing, to determine if the sequence of the primers and probes is correct.

The following examples are exemplary embodiments of the disclosure. One of ordinary skill in the art will understand that slight variations or substitutions may be made to achieve the same results. Those slight variations and substitutions are considered a part of the disclosure herein.

### EXAMPLES

### Example 1

### Recovery of Large Amounts of DNA from Large Volumes of Urine Improve ctDNA Assay Sensitivity

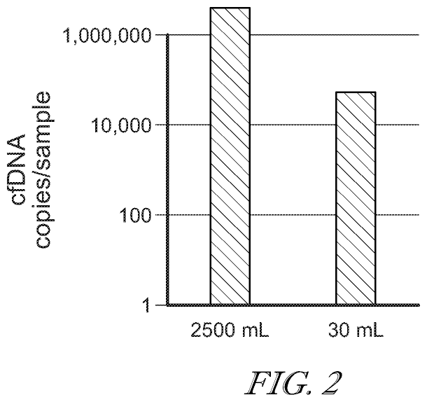

Analysis of large volumes of urine is challenging because of the lack of appropriate commercial DNA extraction solutions. As DNA extraction introduces small amounts of PCR inhibitors one cannot simply subdivide a large urine sample into 100 small ones and use routine extraction methods. The data suggest that crossflow (or tangential flow) diafiltration using polyethersulfone (PES) membranes (e.g., 0.1 m2 surface, ‘5 kDa’ pore size, Sartorius, Germany) are suitable to simultaneously concentrate and diafilter up to 3000 mL urine down to a few mL, which can then be extracted using standard commercial DNA extraction kits (Norgen, Canada). In one example, a 77-fold ddPCR signal increase was observed when comparing 2500 mL (using PES membrane diafiltration prior to DNA extraction) vs. 30 mL of the same urine, while an 83-fold signal increase was theoretically possible based on volume ratio (93% efficiency) (see FIG. 2).

### Example 2

### Novel Method to Elongate Small DNA Fragments for use in Standard ctDNA Mutation Quantifying Assays

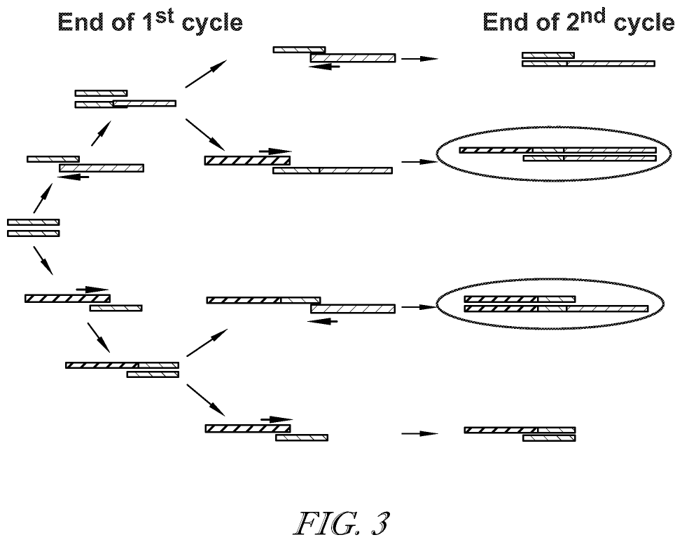

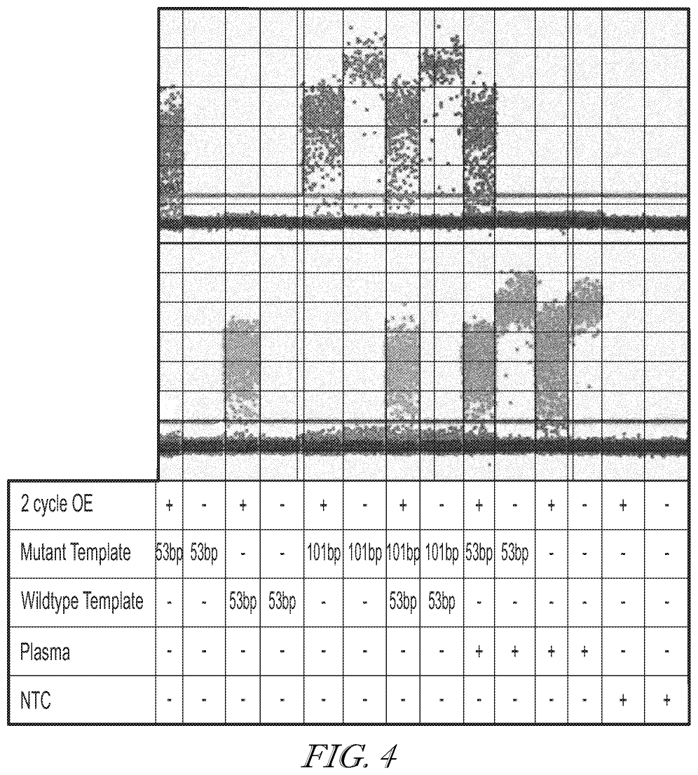

Fragment length distribution analysis of fetal transrenal DNA suggests that there are 10 to 100 times more transrenal DNA molecules of 30-60 base pair (bp) length than there are of those of 100 bp length. However, mutations in small (e.g., 30-60 bp) DNA fragments are not easily quantifiable using standard methods. Fragments below 60 bp cannot be detected with a typical probe based ddPCR mutation assay and are not suitable for routine ctDNA NGS assays. Fragments over 100 bp can be reliably detected using a 100 bp ddPCR assay and are suitable for NGS studies. The efficiency of DNA fragment detection between ˜60 and ˜90 bp is assay specific but typically low. The potential signal gain that can be achieved by quantifying mutations in DNA fragments 30-60 bp can be estimated as the ratio of the amount of ctDNA from 30 to 60 bp in length over the amount of longer ctDNAs. Based on fetal transrenal DNA fragment length distribution, possible signal gain factors of far over 100 are likely, suggesting that mutation detection across a range of small transrenal DNA fragments could dramatically increase sensitivity of ctDNA assays (even independent from large volume urine DNA recovery). An overlap extension (OE) PCR-based DNA fragment elongation method (FIG. 3, 4) was developed. Briefly, two PCR cycles using extension primers, each 45-55 bp long are sufficient to elongate fragments of interest while also limiting PCR-errors. Targeting very short 30-60 bp DNA fragments with both primers having 15 bp or more overlap with the DNA template allows assay design (extension primers centered on mutation flanking the ddPCR probe site) and specific amplification for virtually all single nucleotide alterations (SNAs). Given that the exact DNA fragment sizes harboring a mutation of interest are unknown in a clinical sample, extension primers are designed using the gene's native DNA sequence to extend a wide range of short DNA fragments. Importantly, this will allow running the OE PCR without knowledge about which fragment sizes are present, and most fragments with >15 bp primer overlap will be elongated. This approach will not limit analysis to a specific DNA fragment length. Fragments of interest with significantly less than 15 bp overlap cannot be extended with this method. The data suggests that the OE PCR works reliably in a complex background, such as that found in plasma or urine (FIG. 4, 5).

OE primers:
Forward:
GTCCTTAGTGATATTGACCAGAGTTTCAACAAAGTAGCTGAA
Reverse:
GTGCCTTCTTCCACTCCTTTCAGTTTCTCTTGTAGTCTCATTAAGA
Fragments used for figure:
Mut Short (53 bp)
CAAAGTAGCTGAATGTGTCTTAATGAGACTACAAGAGA
Mut Long (101 bp)
GTCCTTAGTGATATTGACCAGAGTTTCAACAAAGTAGCTGAATGTGTCT
TAATGAGACTACAAGAGAAACTGAAAGGAGTGGAAGAAGGCAC
DdPCR primers:
Forward:
TCCTTAGTGATATTGACCAGAGTTT
Reverse:
TGCCTTCTTCCACTCCTTTC
Mutant probe:
GCTGAATGTGTCT
Wildtype probe:
GCTGAACGTGTCT

### Example 3

### Two-Pronged Approach to Increase the Sensitivity of Urine-Based ctDNA Detection

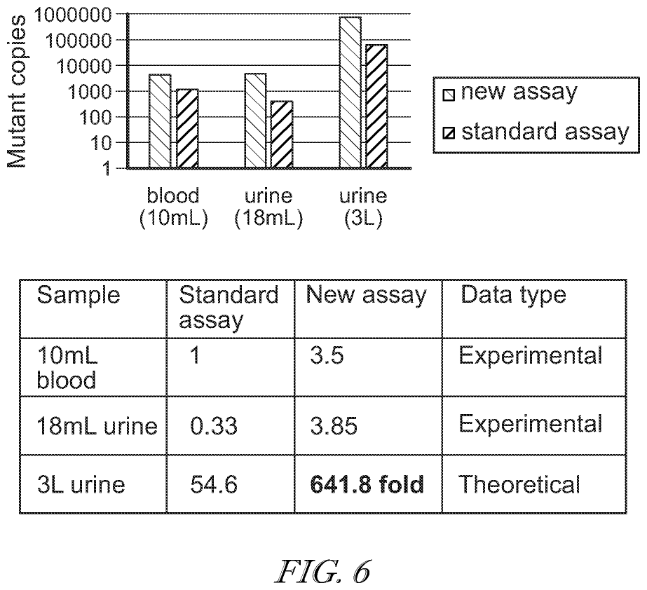

Matched blood and urine samples were analyzed to determine the potential overall signal gain when combining both approaches: (a) 24 hour/3 L urine vs. one time 10 mL blood collection and b) using short DNA fragment (e.g., 30-60 bp) mutation analysis vs. standard length DNA fragment (e.g., 100-200 bp) mutation analysis alone. The data suggest that analysis of 24-hour urine using our short DNA fragment assay could produce a >500-fold signal gain over a standard mutation assay analyzing 10 mL blood (FIG. 6).

### Example 4

### Extracting DNA from Large Volumes of Urine

The capability to extract DNA from large volumes of urine suitable for downstream mutation analysis was developed. No solutions for DNA extraction from very large volumes of urine suitable for routine rare mutation analysis methods previously existed. A scalable method was developed. To clearly demonstrate the signal gain associated with the analysis of large urine volumes over small volume urine analysis, identical DNA quantification assays (i.e. same standard ddPCR assay for the same mutation) were performed on matched samples. DNA extracted from a 24-hour urine samples using the optimized protocol as herein described produced about a 20-fold higher ctDNA signal than DNA from about a 30 mL urine sample. The data show that large volume urine-based ctDNA detection is feasible and identifies more mutant copies than small urine sample ctDNA analysis.

### Example 5

### Patient Selection

The following inclusion criteria was used for all patients: (i) informed consent, (ii) non-genitourinary solid tumor with pathologically confirmed cancer diagnosis, (iii) advanced disease with an estimated 20 mL or greater of total tumor volume. The following exclusion criteria will be used: (i) reduction in glomerular filtration rate <30 mL/min, (ii) genitourinary (GU) tract metastasis. Feasibility: Patients with typically widely metastatic disease commonly will have clinical mutation testing data available, which was used to identify patient-specific mutations for assay development. Most patients will be recruited when they present for evaluation for palliative radiation therapy. There is a highly functional departmental clinical research infrastructure in place for recruitment and sample collections.

### Example 6

### Urine and Blood Collection and Preparation

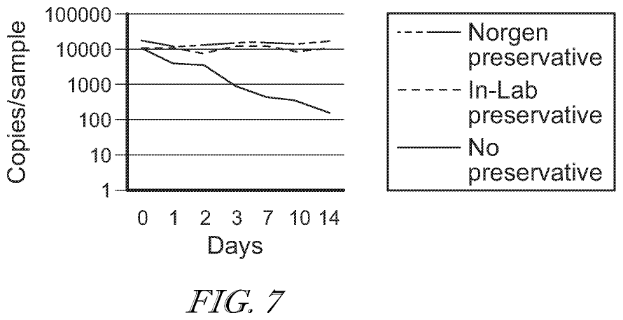

Similar to very common 24-hour urine laboratory tests performed for various reasons (e.g., kidney stone or endocrine work-up) study patients were provided with a 3 L urine collection container (URISAFE, Simport Scientific), which contained about 60 mL of urine DNA preservative (Norgen, Canada) and was sufficiently large for a 24-hour urine collection for >95% of patients. Based on preliminary studies, ctDNA remained stable for several days if using a commercial urine ctDNA preservative (Norgen, FIG. 7). Patients were also provided a urine collection insert (Gent-L-Pan) that can be placed on a toilet to facilitate collection. Study participants were instructed to collect a 24-hour urine sample. Creatinine measurements will identify significant noncompliance (i.e. only 8-hr urine collection or less). A matched 20 ml blood sample is collected in two cfDNA preservation tubes (Streck, Nebr.), which occur during an office visit and typically coincide with the beginning or end of the 24-hour urine collection. In addition, available clinical ctDNA test results are reviewed. Urine cfDNA was extracted using the Urine Cell-Free ctDNA Purification Maxi kit (Norgen). For blood, the QiAamp Circulating Nucleic Acid Kit (Qiagen, Germany) is used.

### Example 7

### Spike in Oligonucleotide Controls

DNA sequences from the Sulfolobus turreted icosahedral virus (STIV), known to not have homology with the human genome, only existing in hot springs, and not able to colonize humans are used. Various STIV control sequences of different lengths (incl. 35, 150, 250, 304 bp) were spiked in the preservative prior to collection, and then in the collected unprocessed urine sample at defined amounts and were quantified at various stages as controls and to calculate efficiencies of sample processing steps.

### Example 8

### Mutation Identification

Clinical mutation testing data was reviewed to identify SNAs. Mutations were prioritized for biofluid testing according to their mutant allele frequency. The presence of 1-3 SNAs in plasma from 10 mL of blood (reserving the other 10 mL blood) was tested using mutations detected in blood for assay development, facilitating quantitative signal gain determinations. In some cases without clinical mutation testing data available, exome sequencing on FFPE biopsy tissue using buffy coat germline DNA as a reference to identify cancer specific mutations was conducted.

### Example 9

### Digital Droplet PCR (ddPCR)

Described herein are ddPCR assay design, assay validation, and conduct of studies (QX200, BioRad) [19]. Probe-based ddPCR assays were employed to quantify mutations in biofluids in triplicates whenever indicated. ddPCR was employed for quality control (QC) assays, to verify performance of individual steps in the workflow. ddPCR Evagreen assays, allowing quantification of very short DNA fragments but are not mutation specific, were used to quantify the amounts of the spiked in control STIV sequences prior to and after PES membrane diafiltration. In addition, for DNA quality control, our custom ddPCR Evagreen ALB QC assays generating 33, 58, 90, and 150 bp amplicons will be used to assess sample degradation at various processing steps. In other embodiments cfDNA sequencing or ddPCR may be used. In another embodiment, ddPCR is preferred to avoid lengthy analysis common for sequencing-based approaches.

### Example 10

### Three-Pronged Approach for Large Volume Urine ctDNA Analysis

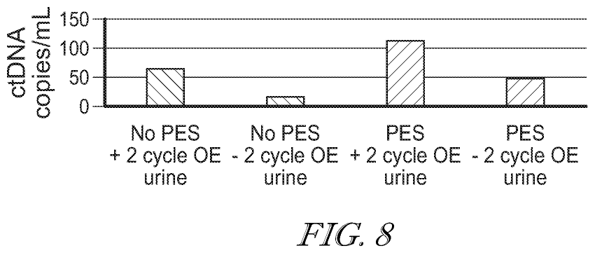

Urine crossflow diafiltration: Crossflow diafiltration combines dialysis for removal of soluble PCR inhibitors with concentration of urine prior to cfDNA extraction (FIG. 8). The optimal polyethersulfone (PES) membrane (Sartorius, Germany) pore sizes (e.g., 3 or 5 kDa) was identified to maximize recovery of about 30-60 bp DNA fragments without significant loss of about 100-200 bp fragments from about 3 L urine. Evagreen ddPCR assays for the ubiquitous wildtype ALB gene, expected to be present in all human urine samples, was used in addition to STIV spike in controls to compare the recovery of 30-60 bp fragments as well as retention of 100-200 bp fragments amongst various methods during the development process. Moreover, alterations in crossflow diafiltration such as addition of about 2 L deionized water after concentration further improves DNA desalting, overcoming PCR inhibition. Presence of PCR inhibition is defined as an at least 25% relative reduction in the normalized ddPCR count increase comparing 1 to 2 vs. 2 to 4 times the input DNA amounts.

Overcoming PCR inhibitors introduced during DNA extraction: Most commercial DNA extraction solutions employ buffers that introduce PCR inhibitors during the extraction process, which interfere with ultra-high sensitivity downstream mutation analyses. To address this, the optimal concentration of PCR additives was determined, such as BSA (e.g., 0.2-0.5 ug/ul) to overcome extraction-based PCR inhibition and maximize ctDNA signal.

Enrichment of transrenal DNA: DNA found in urine consists of both transrenal and non transrenal DNA fractions. DNA from the genitourinary (GU) tract is usually vastly more abundant in urine than transrenal DNA. Thus, presence of GU DNA leads to a significant reduction in mutant allele frequencies, making the quantification of already low frequency alterations even more difficult. To enrich for ctDNA found only in transrenal DNA, transrenal DNA is typically short (about <120 bp), with a peak around about 30-60 bp while the majority of GU tract DNA is >200 bp. Size selection methods were tested (including commercially available column-based solutions) to eliminate the fraction of longer GU DNA fragments using STIV spike-ins for amplicons of 35 bp, 150 bp and 250 bp. The size selection method chosen for evaluation is the one that retains the most 35 bp and 150 bp fragments (highest combined signal) while excluding at least 99% (if not achievable 95% or 90%) of the 250 bp fragments.

Determining the ctDNA signal gain of 1-3: Using the previously described methods for the three-pronged approach, the ctDNA signal gain was determined using 24-hour urine over 30 mL urine in the study cohort (see Example 5).

### Example 11

### Alternative Approaches

The proposed approach lead to an at least 20-fold ctDNA signal increase over a 30 mL urine ctDNA assay. A patient population with a large ctDNA burden was selected to ensure all biospecimens (incl. blood and 30 mL urine) are positive for ctDNA to facilitate quantitation of signal gains across samples. A retrospective review of cases at the end of the study was performed to exclude patients that developed GU tract metastases after enrollment to prevent interpretation of data skewed by analysis including non transrenal ctDNA. In case PES membranes do not perform well enough, other materials, such as highly hydrophilic Hydrosart membranes (Sartorius, Germany) were explored for combined dialysis and concentration approaches.

### Example 12

### Develop the Capability to Detect Mutations in Very Small DNA Fragments

The overwhelming majority of transrenal DNA fragments in urine are very short (e.g., 30-60 bp) and cannot be readily assayed using routine mutation quantification approaches. The objective of this disclosure is to provide a novel method for allowing routine quantification of mutations in such very short DNA fragments and determine the signal gain associated with very short DNA fragment (e.g., 30-60 bp) analysis over longer DNA fragments (e.g., 100-200 bp). Urine mutation analysis of about 30-60 bp DNA fragments will produce a ctDNA signal at least 5-fold higher than the signal from DNA fragments longer than about 100 bp. This disclosure provides evidence that analysis of short DNA fragments result in a significant ctDNA signal gain.

### Example 13

### Methodology and Experimentation for Analyzing Mutations

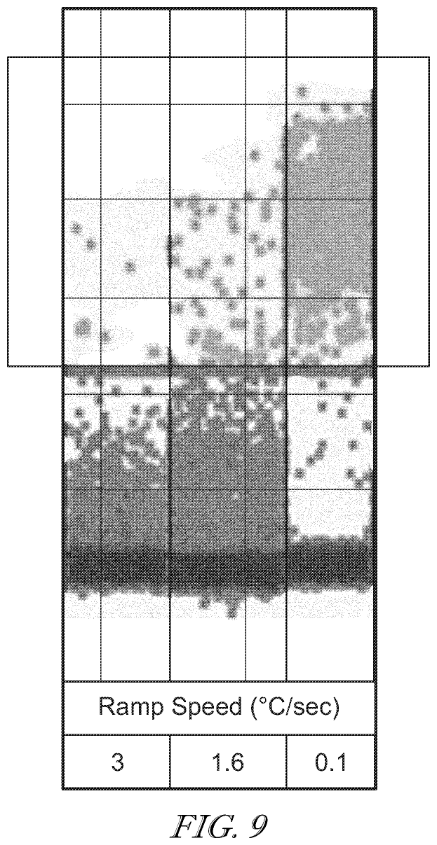

Eligibility and urine collection will be as described above for Examples 1 through 12. A 30 ml urine aliquot was removed from 24-hour urine for DNA extraction, size selected, and tested for the presence of SNAs for assay development. For OE PCR QC, a 35 bp spike-in STIV oligonucleotide undergo a control OE PCR with separate extension primers in parallel to all OE PCRs for ctDNA elongation. ddPCR Evagreen assays were used to quantify the amounts of original 35 bp oligonucleotides and its elongated 80 bp product. Their ratio is a measure of the efficiency of the OE PCR. Variables were identified that do not appear to have a significant impact on efficiency such as the specific Taq polymerase used. However, some alterations of OE PCR conditions (e.g., reducing ramp speed to about 0.1 C./s, FIG. 9 or increasing primer concentration during ddPCR)) appear to have great potential to improve efficiency. Thus, how alterations in PCR parameters including primer concentration, concentration of PCR additives, PCR temperatures, and temperature ramp speeds contribute to maximizing elongation efficiency were analyzed. Moreover, if emulsion PCR can overcome possible OE PCR bias for elongation of ultra-low frequency mutations was analyzed. The elongation efficiency under various PCR conditions was determined using in vitro systems of varying mutant and wildtype DNA fragment sizes, modeling human urine samples with varying DNA fragment lengths. Templates designed with varying overlap of about 10-25 bp with OE primers to identify a minimum overlap length required for successful elongation at various temperatures. After identifying optimal conditions for OE PCR, emulsion PCR was used to estimate the false positive rate by testing 10⁶ PCR negative control reaction volumes (one 96-well plate). The ctDNA signal gain associated with the use of elongation PCR in urine samples from a cohort of patients with advanced solid tumors was then determined (Example 5).

### Example 14

### Alternative Approaches to Analyzing Mutations

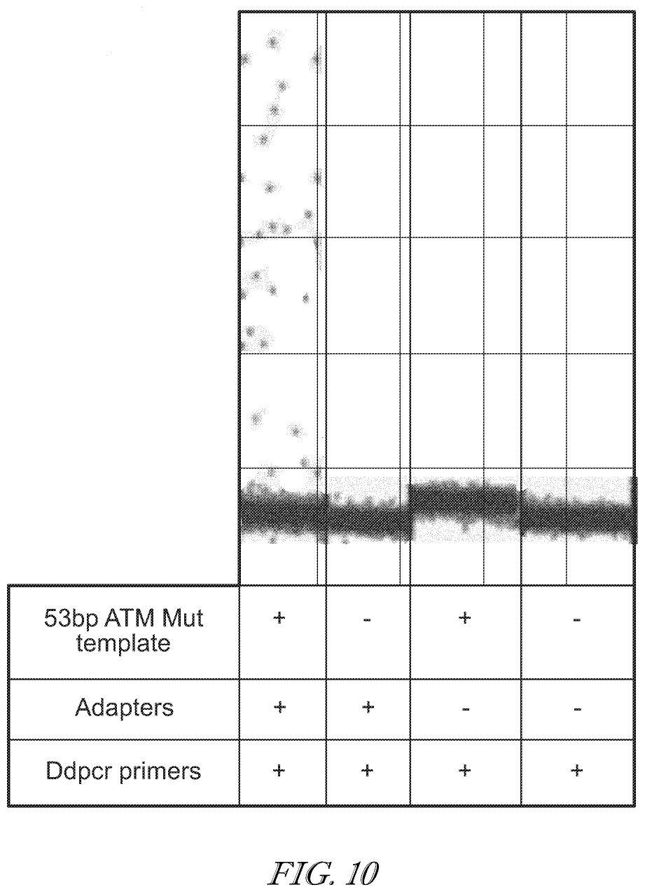

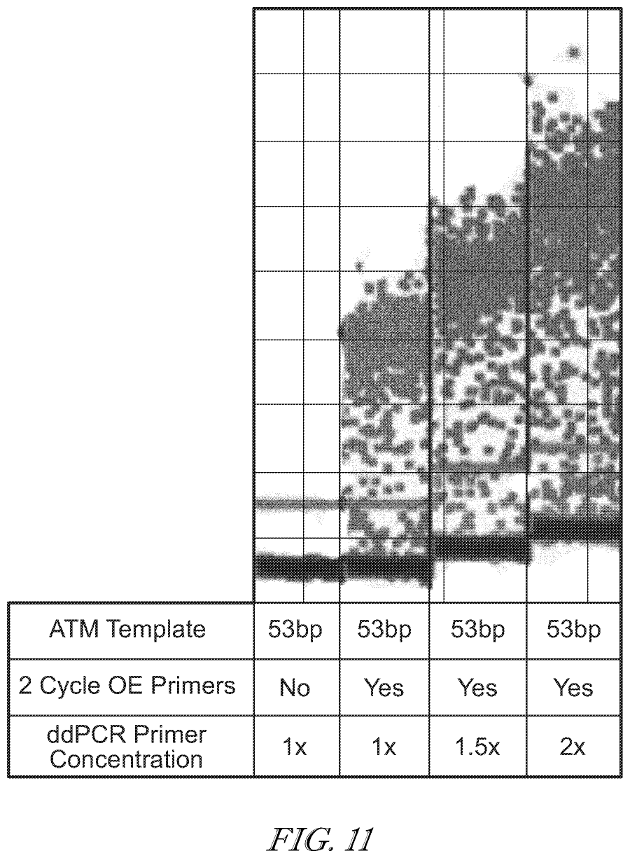

Short fragment ctDNA quantification based on OE PCR of about 30-60 bp fragment elongation resulted in an at least 5-fold urine ctDNA signal increase over analysis of about 100-200 bp DNA fragments. A PCR-free ligation-based elongation assay using T4 ligase was tested (FIG. 10), which was used as an additional control or alternative approach to demonstrate the ctDNA signal gain associated with small DNA fragment analysis. Alternatively, single-stranded DNA ligation protocols were used to capture sub-100 bp DNA fragments. Also, efficiency of mutant fragment elongation can be enhanced by preincubation with wildtype blocking oligonucleotides and related approaches. FIG. 11 shows that primer increase during ddPCR increases separation between mutant (blue) and control signal (black bands above x-axis).

### Example 15

### Detection of Low Mutant Allele Fraction (MAF)

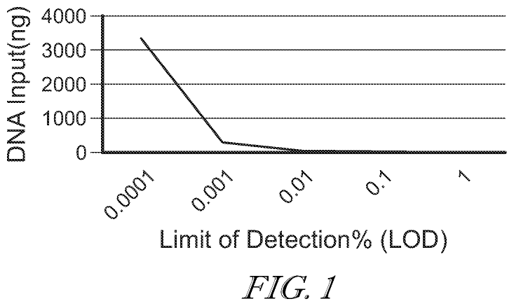

Detection of low mutant allele fraction (MAF) ctDNA is limited by the amount of input DNA (FIG. 1), which is a function of the amount of blood sampled. Typically, 10 or 20 mL of blood are drawn for a ctDNA test. Strategies have been explored to increase the sensitivity of cancer mutation detection in blood. For example, combined analysis of DNA and RNA can result in increased sensitivity over ctDNA analysis alone. Similarly, monitoring of several mutations results in increased sensitivity over monitoring of just one mutation. Moreover, certain tumor features beyond tumor size (Table 1), such as proliferation index, metabolic activity and histology may be important factors that determine the probability of ctDNA detection.

Throughout the specification, reference is made to various references. Each is incorporated herein by reference in its entirety.

### REFERENCES

1. Bettegowda, C., et al., Detection of circulating tumor DNA in early-and late-stage human malignancies. Sci Transl Med, 2014. 6(224): p. 224ra24.

2. Newman, A. M., et al., An ultrasensitive method for quantitating circulating tumor DNA with broad patient coverage. Nat Med, 2014. 20(5): p. 548-54.

3. Abbosh, C., et al., Phylogenetic ctDNA analysis depicts early-stage lung cancer evolution. Nature, 2017. 545(7655): p. 446-451.

4. Chused, T. M., A. D. Steinberg, and N. Talal, The clearance and localization of nucleic acids by New Zealand and normal mice. Clin Exp Immunol, 1972. 12(4): p. 465-76.

5. Yu, S. C., et al., High-resolution profiling of fetal DNA clearance from maternal plasma by massively parallel sequencing. Clin Chem, 2013. 59(8): p. 1228-37.

6. Diamandis, E. P. and C. Fiala, Can circulating tumor DNA be used for direct and early stage cancer detection? F1000Res, 2017. 6: p. 2129.

7. Tsui, N. B., et al., High resolution size analysis of fetal DNA in the urine of pregnant women by paired-end massively parallel sequencing. PLoS One, 2012. 7(10): p. e48319.

8. Merker, J. D., et al., Circulating Tumor DNA Analysis in Patients With Cancer: American Society of Clinical Oncology and College of American Pathologists Joint Review. J Clin Oncol, 2018: p. JCO2017768671.

9. Chaudhuri, A. A., et al., Early Detection of Molecular Residual Disease in Localized Lung Cancer by Circulating Tumor DNA Profiling. Cancer Discov, 2017. 7(12): p. 1394-1403.

10. Krug, A. K., et al., Improved EGFR mutation detection using combined exosomal RNA and circulating tumor DNA in NSCLC patient plasma. Ann Oncol, 2017.

11. Tchekmedyian, N., et al., Longitudinal monitoring of ctDNA EGFR mutation burden from urine correlates with patient response to EGFR TKIs: A case series. Lung Cancer, 2017. 108: p. 22-28.

12. Franovic, A., et al., Urine test for EGFR analysis in patients with non-small cell lung cancer. J Thorac Dis, 2017. 9(Suppl 13): p. S1323-S1331.

13. Husain, H., et al., Monitoring Daily Dynamics of Early Tumor Response to Targeted Therapy by Detecting Circulating Tumor DNA in Urine. Clin Cancer Res, 2017. 23(16): p. 4716-4723.

14. Reckamp, K. L., et al., A Highly Sensitive and Quantitative Test Platform for Detection of NSCLC EGFR Mutations in Urine and Plasma. J Thorac Oncol, 2016. 11(10): p. 1690-700.

15. Chen, S., et al., Urinary circulating DNA detection for dynamic tracking of EGFR mutations for NSCLC patients treated with EGFR-TKIs. Clin Trans! Oncol, 2017. 19(3): p. 332-340.

16. Wang, X., et al., Investigation of transrenal KRAS mutation in late stage NSCLC patients correlates to disease progression. Biomarkers, 2017. 22(7): p. 654-660.

17. Siravegna, G., et al., Tracking a CAD-ALK gene rearrangement in urine and blood of a colorectal cancer patient treated with an ALK inhibitor. Ann Oncol, 2017. 28(6): p. 1302-1308.

18. Fujii, T., et al., Abstract 3146: Circulating tumor DNA assay performance for detection and monitoring of KRAS mutations in urine from patients with advanced cancers. Cancer Research, 2016. 76(14 Supplement): p. 3146-3146.

19. Deig C R, et al., In Vitro Cell-free DNA Quantification: A Novel Method to Accurately Quantify Cell Survival after Irradiation. Radiat Res., 2018. 190: p. in press.

20. Burnham, P., et al., Single-stranded DNA library preparation uncovers the origin and diversity of ultrashort cell-free DNA in plasma. Sci Rep, 2016. 6: p. 27859.

21. Gansauge, M. T. and M. Meyer, Single-stranded DNA library preparation for the sequencing of ancient or damaged DNA. Nat Protoc, 2013. 8(4): p. 737-48.

22. Song, C., et al., Elimination of unaltered DNA in mixed clinical samples via nuclease-assisted minor-allele enrichment. Nucleic Acids Res, 2016. 44(19): p. e146.

1. A method of detecting or monitoring circulating tumor DNA (ctDNA) in a patient urine sample comprising:
i. processing a patient urine sample to concentrate the sample;
ii. extracting the ctDNA from the sample; and
iii. analyzing the ctDNA in the sample wherein the ctDNA comprises short DNA fragment of less than 100 base pair (bp) in length.

2. The method of claim 1, wherein the processing step comprises filtering the sample, dialyzing the sample, or combinations thereof

3. The method of claim 1, wherein the processing step comprises urine crossflow diafiltration.

4. The method of claim 1, further comprising neutralizing PCR inhibitors in the sample.

5. The method of claim 1, further comprising removing the non-transrenal DNA from the sample.

6. The method of claim 1, wherein processing the patient urine sample and extracting the ctDNA occur in the same step.

7. The method of claim 1, wherein the urine sample is a sample that has been collected from a patient for about 24 hours.

8. The method of claim 1, wherein the DNA is extracted using a size selectivity method.

9. The method of claim 1, wherein the ctDNA is analyzed for mutations.

10. The method of claim 9, wherein the mutation indicates a disease state in the patient.

11. The method of claim 10, wherein the disease is cancer.

12. The method of claim 10, wherein the disease is non-small cell lung carcinoma.

13. The method of claim 9, wherein the ctDNA is analyzed by PCR.

14. The method of claim 13 wherein the PCR is overlap extension PCR (OE PCR).

15. The method of claim 13 wherein the PCR is digital droplet PCR (ddPCR) or emulsion PCR (EmPCR).

16. The method of claim 1, wherein the ctDNA is analyzed for the presence of a tumor marker or a tumor recurrence marker.

17. The method of claim 1, comprising monitoring the patient for cancer progression.

18. The method of claim 1, comprising determining if the patient is eligible for a targeted cancer therapy.

19. The method of claim 11, wherein the cancer is a non-metastatic cancer.

20. The method of claim 1, wherein the extracted ctDNA comprisises short DNA fragment of about 30 to about 60 bp in length.
